(19) United States
(12) Patent Application Publication
(10) Pub. No.: US 2018/0299440 A1
(43) Pub. Date: Oct. 18, 2018

(54) HIGH-DENSITY WESTERN BLOT ARRAY ANALYSIS METHOD

(71) Applicant: T-MAC CO., LTD., Daejeon (KR)

(72) Inventors: Sung Hyun KANG, Daejeon (KR); Tae Sung YOON, Daejeon (KR)

(73) Assignee: T-MAC CO., LTD., Daejeon (KR)

(21) Appl. No.: 16/010,031

(22) Filed: Jun. 15, 2018

Related U.S. Application Data
(63) Continuation of application No. PCT/KR2016/014836, filed on Dec. 16, 2016.

(30) Foreign Application Priority Data
Dec. 18, 2015 (KR) 10-2015-0181680
Dec. 16, 2016 (KR) 10-2016-0172742

(51) Int. Cl. G01N 33/561; G01N 27/447; G01N 33/543

(52) U.S. Cl. CPC G01N 33/561; G01N 33/543; G01N 27/44739

(57) ABSTRACT

The present invention relates to a high-density western blot array analysis method capable of simultaneously checking whether specific proteins are present in a plurality of samples, or simultaneously checking whether a plurality of proteins are present in a single sample, and according to the analysis method of the present invention, there are advantages of: reducing sample volume; enabling analysis even with a very small amount of antibodies; enabling the time required for testing to be remarkably shortened and a device to be compactly formed without high-priced equipment; and enabling analysis to proceed without restrictions on test formats.

### TECHNICAL FIELD

The present invention relates to a high-density western blot array analysis method, and more particularly, to a high-density western blot array analysis method capable of simultaneously checking whether specific proteins are present in a plurality of samples, or simultaneously checking whether a plurality of proteins are present in a single sample.

### BACKGROUND ART

Western blotting is a research technique for detecting expressed proteins through analysis of a specific sample.

Western blotting is used to detect the presence or the post-translational modification of proteins in cells or tissues.

Roughly, the process of western blotting is performed in the order of SDS-PAGE, protein transfer to a nitrocellulose or PVDF transfer membrane, treatment with an antibody against a target protein, and detection of the target protein through antibody labeling analysis. As samples for western blotting, generally, protein mixtures from cells or tissue are used. Before use in western blotting, a sample is prepared by cell lysis, and then proteins are purified through several times of centrifugation and other separation methods.

To effectively perform the detection of target proteins with a very small amount, it is necessary to widely spread proteins in the sample, and to this end, sodium dodecyl sulfate-polyacrylamide gel electrophoresis (SDS-PAGE) is frequently used. SDS binds to a protein in a sample such that the proteins lose secondary, tertiary and quaternary structures and become linear, and a high density of negative charges are imparted to the protein to be attracted towards the positive electrode in electrophoresis. That is, SDS-PAGE as described above is for sorting proteins denatured with SDS by size on a polyacrylamide gel (PAG) through electrophoresis.

Since the proteins separated by SDS-PAGE are buried inside of the gel, observation of direct binding between a probe and the protein is difficult.

Therefore, the protein is transferred onto a thin transfer membrane to facilitate binding of a probe to the protein.

Among a variety of such transfer methods, the most widely used method is an electrophoretic transfer method.

This method uses a very similar principle to electrophoresis, and two methods have been conventionally used.

One is a semi-dry transfer method for transferring proteins by placing a gel horizontally, and the other is a wet transfer method for transferring proteins while a gel is vertically placed in a tank filled with buffer.

Here, in the transfer method, after the electrophoresis, the gel is carefully separated, and placed onto the transfer membrane.

Then, filter papers and pads are placed at both sides of a gel and transfer membrane to sufficiently absorb a buffer and to protect the gel, and a negative electrode and a positive electrode are placed at both sides thereof.

Here, since a protein is attracted to the positive electrode, the transfer membrane is placed at the positive electrode side.

When a voltage is applied, proteins gradually begin to be transferred from the gel to the membrane, resulting in attachment onto the transfer membrane.

When the proteins are transferred onto the transfer membrane, a blocking process is performed to prevent attachment of other proteins to the transfer membrane.

This blocking process allows proteins which do not react with previously known probes to be attached to all sites except those to which the sample proteins are attached.

The probe antibody is produced from lymphocytes, and has a property of specifically binding only to a target protein.

That is, the probe needs to be previously prepared for an experiment, and different types of probes should be used depending on target proteins.

Therefore, conventional western blotting had problems of excessive consumption of required antibodies and demand for an excessive amount of a sample.

As a result, there was a limit to the number of samples used at once.

Moreover, a conventional western blotting system requires much time consumption for electrophoresis, and a long electrophoretic distance for migration of a sample.

Meanwhile, in U.S. Unexamined Patent Application Publication No. 2011-0028339 as prior art, a highly-integrated western blotting technique is disclosed.

However, U.S. Unexamined Patent Application Publication No. 2011-0028339 has many problems, for example, a demand for high-priced equipment, and difficulty in freely configuring test formats, particularly, limitation of the number and types of samples that can be tested at one time.

(Patent Document 1) U.S. Pat. No. 8,940,232 B2

(Patent Document 2) U.S. Pat. No. 7,670,833 B2

(Patent Document 3) US2011-0028339 A1

### DISCLOSURE

### Technical Problem

One object of the present invention is to overcome the limitation of a conventional western blotting, and to implement a highly-integrated array technique for performing multiple western blotting simultaneously.

In addition, another object of the present invention is to provide gel formers, a sample injection unit and antibody incubator chambers, which are necessary for implementing the above-described technique.

### Technical Solution

To achieve the above-mentioned objects, one aspect of the present invention provides a high-density western blot array analysis method, which includes: preparing a gel in which a plurality of sample injection holes are arranged in one or more rows; injecting a sample into each sample injection hole in the gel; electrophoresing the sample-injected gel in a direction perpendicular to the row of the sample injection holes; transferring the sample in the electrophoresed gel to a membrane; putting the entire sample-transferred membrane into one area formed in an antibody incubation chamber and treating the membrane with an antibody; and analyzing the antibody-treated membrane.

To achieve the above-mentioned objects, another aspect of the present invention provides a high-density western blot array analysis method, which includes: preparing a gel in which one or more sample injection slots each having a predetermined width are arranged in a longitudinal direction of the width so as to form one or more rows; injecting a sample into the sample injection slots in the gel; electrophoresing the sample-injected gel in a direction perpendicular to the array of the sample injection slots; transferring the samples in the electrophoresed gel to a membrane; placing the sample-transferred membrane, in an antibody incubation chamber in which a plurality of antibody injection holes and a plurality of microchannels are formed to fit the width of the sample injection slots, and then treating the sample on the membrane with antibodies in microchannels by injecting different types of antibodies into the respective antibody injection holes; and analyzing the antibodies-treated membrane.

### Advantageous Effects

According to the present invention as described above, the following effects are exhibited:

First, it is possible to perform high-density western blotting, resulting in the decrease in the volume of a sample;

Second, analysis can be performed even with a very small amount of an antibody;

Third, the number of samples which can be used in one experiment is greatly increased;

Fourth, the time required for the experiment is greatly reduced;

Fifth, an electrophoresis distance is shortened and therefore test instruments become compact;

Seventh, there are no need of high-priced equipment and no limitation of test formats; and

Eighth, since there is a sealing member between an upper frame and a lower frame, an antibody incubation chamber is sealed.

### DESCRIPTION OF DRAWINGS

FIG. 1 is a flowchart illustrating the sequence of steps of a high-density western blot array analysis method according to the present invention.

FIGS. 2A to 2C are set of diagrams of gels which are used in a high-density western blot array analysis method according to a first analysis method of the present invention.

FIGS. 3 and 4 are a perspective view and a front view of a first gel former which is used in the high-density western blot array analysis method according to a first analysis method of the present invention, respectively.

FIG. 5 is a front view of a sample injection unit which is used in the high-density western blot array analysis method according to the first analysis method of the present invention.

FIGS. 6A and 6B are a set of perspective views of a first antibody incubation chamber which is used in the high-density western blot array analysis method according to the first analysis method of the present invention.

FIG. 7 is a diagram of a gel which is used in a high-density western blot array analysis method according to a second analysis method of the present invention.

FIGS. 8 and 9 are a perspective view and a front view of a second gel former which is used in the high-density western blot array analysis method according to the second analysis method of the present invention, respectively.

FIG. 10A is a perspective view of an example of a second antibody incubation chamber used in the high-density western blot array analysis method according to the second analysis method of the present invention, and FIG. 10B is a perspective view of another example of the second antibody incubation chamber used in the high-density western blot array analysis method according to the second analysis method of the present invention.

FIG. 11A is a cross-sectional view of the cross-section cut along a-a′ of the second antibody incubation chamber according to the first example, and FIG. 11B is a cross-sectional view of the cross-section cut along a-a′ of the second antibody incubation chamber according to the second example.

FIG. 12A is a schematic diagram of an injection unit used in the second antibody incubation chamber according to the first example, and FIG. 12B is a schematic diagram of an injection unit used in the second antibody incubation chamber according to the second example.

FIG. 13A is a schematic diagram of the insertion of the injection unit into the second antibody incubation chamber according to the first example, FIGS. 13B and 13C are schematic diagrams of the insertion of the injection unit into the second antibody incubation chamber according to the second example, in which FIG. 13B showing the cross-section of a region in which injection tubes are inserted, and FIG. 13C showing the cross-section of a region in which discharge tubes are inserted.

### MODES OF THE INVENTION

Hereinafter, the present invention will be described in detail with reference to the accompanying drawings.

In this process, the thicknesses of the lines and the sizes of the components shown in the drawings may be exaggerated for clarity and convenience of explanation. In addition, the following terms are defined in consideration of the functions of the present invention, and this may vary according to the intention or custom of the user or operator. Therefore, the definition of these terms should be based on the contents throughout the specification.

### First Example

One aspect of the present invention provides a high-density western blot array analysis method (first analysis method) for checking whether a protein binding to a single antibody is present in a plurality of samples. The first analysis method is multi sample analysis, for example, may be applied to the analysis using a drug library, a biodrug library, an RNAi library or an antisense RNA library, may be used as an alternative to high throughput screening (HTS) used in in vitro screening, and may be used in screening for various target molecules.

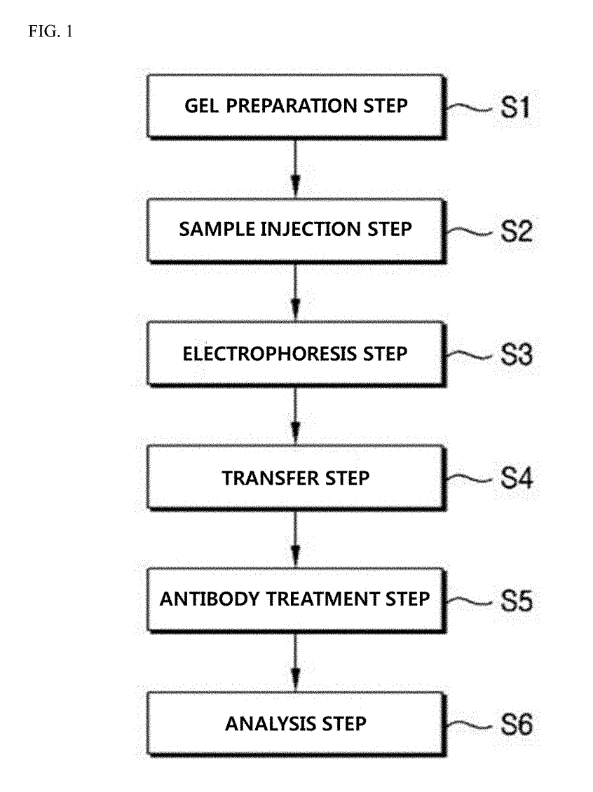

Referring to FIG. 1, the first analysis method of the present invention includes preparing a gel (S1), injecting a sample into the gel (S2), electrophoresing the sample in the gel (S3), transferring the sample in the electrophoresed gel onto a membrane (S4), treating the membrane with an antibody (S5), and analyzing the membrane (S6).
(1-1) Gel Preparation Step (S1)

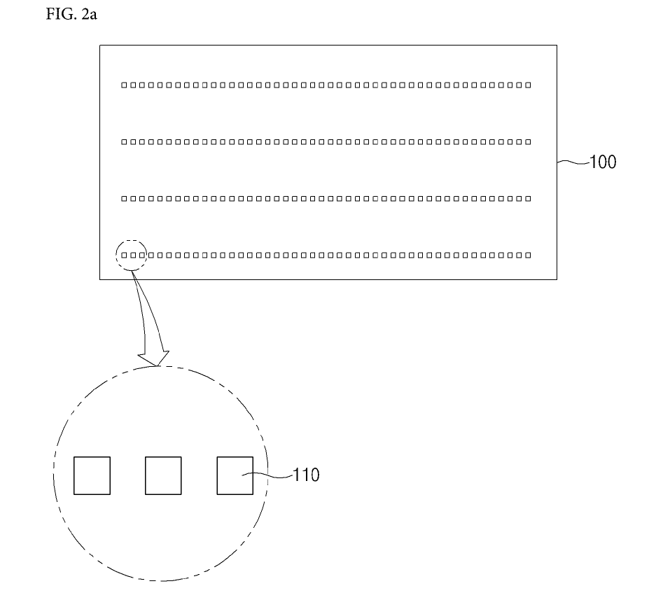

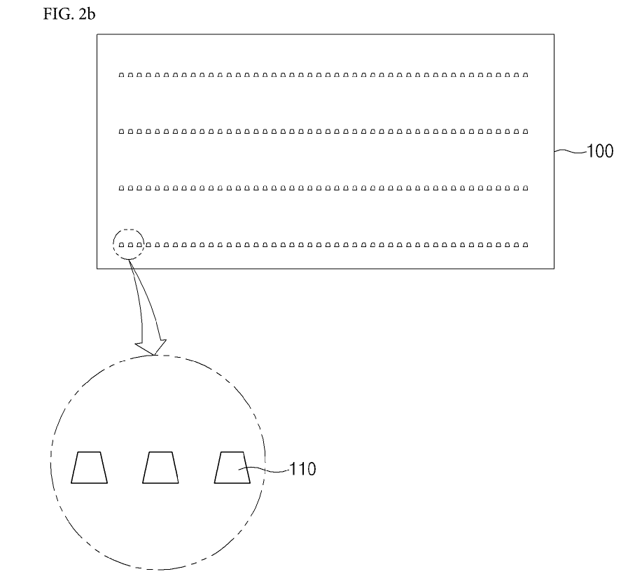

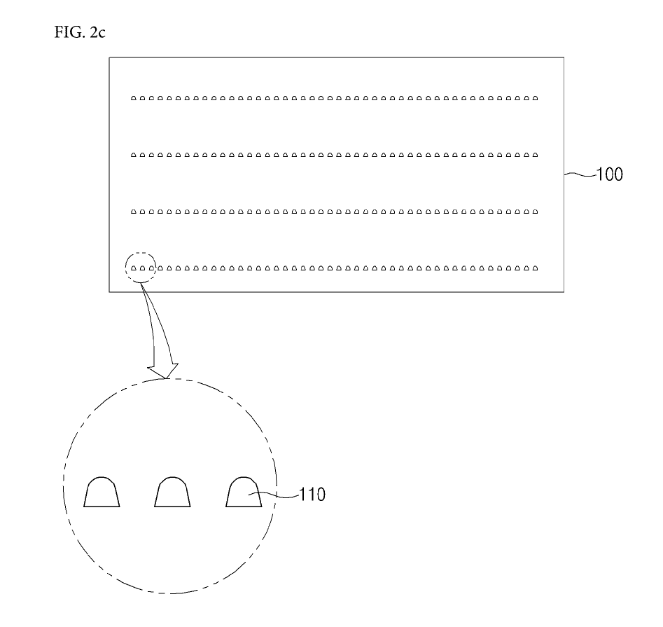

Referring to FIGS. 2A to 2C, in the first analysis method, first, a gel 100 in which a plurality of samples injection holes 110 are formed in one or more rows is prepared (S1).

Each of the plurality of samples injection holes 110 has a size that allows several microliters, preferably, 0.1 to 5 μl, and more preferably 0.5 to 2 μl of a sample to be injected.

20 or more, preferably 50 or more, and more preferably 100 or more of the sample injection holes 110 may be arranged at an interval of several mm, preferably 0.5 to 5 mm, and more preferably 1 to 3 mm to form one row.

In addition, there are a plurality of rows consisting of the plurality of samples injection holes 110. When there are a plurality of rows, the plurality of rows are formed to have an interval larger than the distance at which the sample injected into the sample injection hole is electrophoresed, for example, at an interval of several cm, preferably, 0.5 to 5 cm, and more preferably, 1 to 3 cm.

Specifically, as shown in FIGS. 2B and 2C, the sample injection holes 110 may be tapered in a direction opposite to the direction of electrophoresing the sample, and for example, may be formed in a trapezoidal shape.

In an exemplary embodiment of the present invention, 101 sample injection holes arranged at an interval of 1 mm form one row, and a gel having a size of 12 cm×6 cm in which four rows formed as described above are arranged at an interval of 1.2 cm was prepared.

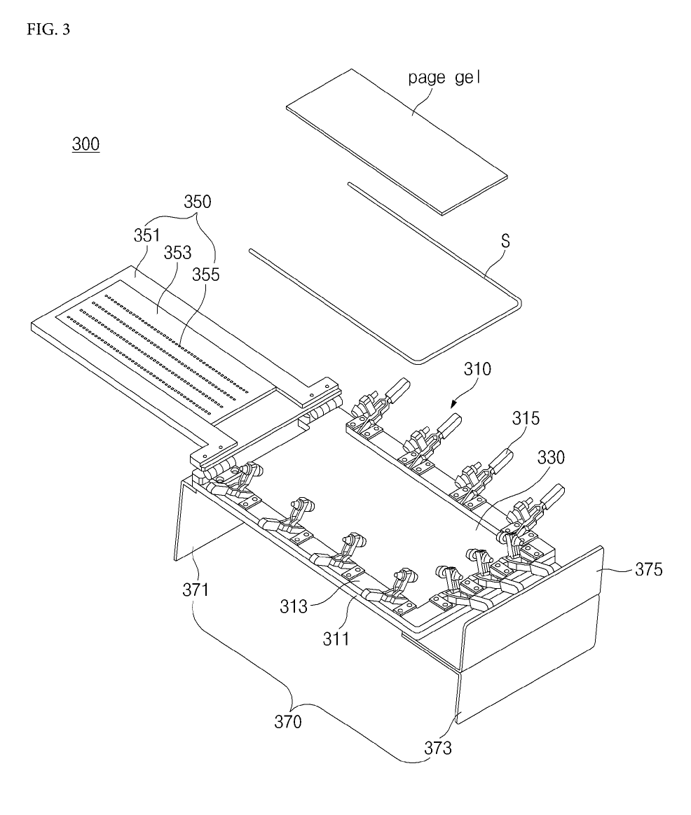

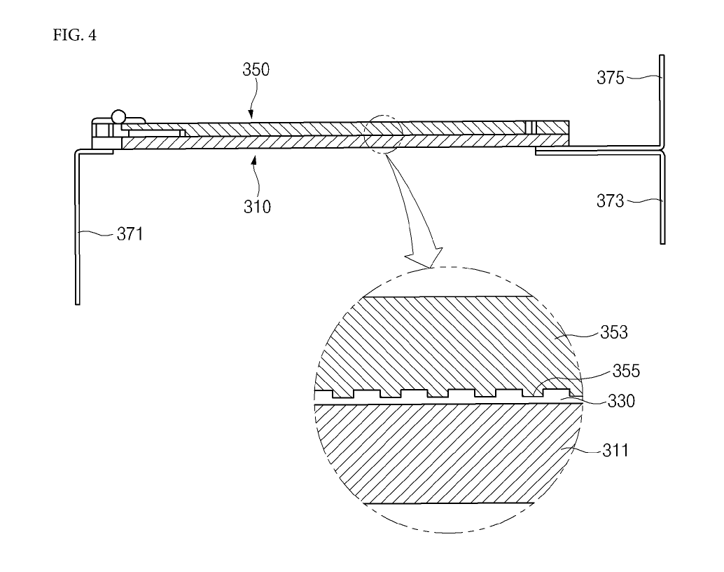

As shown in FIGS. 3 and 4, the gel used in the first analysis method of the present invention may be prepared using a first gel former 300.

Referring to FIGS. 3 and 4, the first gel former includes a main body 310 and a cover unit 350.

The main body 310 includes a first plate 311 formed of a flat plate-shaped member and formed in a rectangular shape, and a second plate 313 formed at an edge of the top surface of the first plate at a predetermined height. The second plate 313 may be formed in a “⊏” shape, and a concave space formed by the flat plate-shaped first plate 311 and the “⊏” shaped second plate 313 becomes a gel container 330 into which gel is injected. As a result, the first plate 311 becomes a bottom surface of the gel container 330, and the second plate 313 becomes a side surface of the gel container 330. Additionally, a sealing member S may be disposed to prevent leakage of the gel out of the gel container 330, and a plurality of clamping apparatuses 315 may be disposed at a predetermined interval on a top surface of the second plate 313.

The cover unit 350 includes a frame 351 and a molding plate 353 including a plurality of molding projections 355. The molding projections 355 are formed so as to protrude from one surface of the molding plate 353, and sample injection holes 110 having a size to allow a volume of several microliters of injected sample are formed by inserting at least some projections into the gel container 330. The molding projections 355 are formed in one or more rows at an interval of several mm, and when there are a plurality of rows consisting of the molding projections 355, the rows may be arranged at an interval of several cm. The molding plate 353 may be integrated with the frame 351, or is more preferably detachable, which is for being replaced according to the pattern, interval, size or number of the plurality of molding projections 355.

The cover unit 350 may be installed to the main body 310 so as to be movable between a first position for opening the gel container 330 and a second position for closing the gel container 330. More preferably, the cover unit 350 may be pivotally coupled to one side of the first plate 311. When the cover unit 350 is disposed at the second position for closing the gel container 330, at least some of the molding projections 355 of the molding plate 353 are inserted into the gel injected into the gel container 330 to make sample injection holes.

While the gel container 330 is sealed by the cover unit 350 at the second position, the cover unit 350 is fixed to the main body 310 by the clamping apparatuses 315. As the sealed state of the gel container 330 by the cover unit 350 using the clamping apparatuses 315 is maintained for a predetermined time, the gel contained in the gel container 330 may be hardened while the sample injection holes 110 corresponding to the shape of the molding projections 355 are formed.

The first gel former 300 may further include a supporting member 370. The supporting member 370 includes a first supporting member 371 and a second supporting member 373, in which the first supporting member 371 supports the bottom of one side of the main body 310, and the second supporting member 373 supports the bottom of the other side of the main body 310. More preferably, the first supporting member 371 is fixed to the lower part of the first plate 311 at a connection side at which the cover unit 350 is pivotably coupled to the main body 310, and the second supporting member 373 is spaced apart from the first supporting member 371 to be fixed to the lower part of the first plate 311 in a longitudinal direction. More preferably, the first supporting member 371 is extended to a direction of the lower part of the first plate 311, and the second supporting member 373 is extended to a direction of the lower and upper parts of the first plate 311. The part that is extended to the lower direction is a second supporting member 373, and the part that is extended to the upper direction is a third supporting member 375. The second supporting member 373 and the third supporting member 375 are formed in a flat plate shape, and support on the ground such that the main body 310 can be set up in the vertical direction. In other words, while the cover unit 350 is fixed with the clamping apparatuses 315 at the second position for closing the gel container 330, the main body 310 may be set up in the vertical direction to the ground by the second supporting member 373 and the third supporting member 375. In addition, since the first supporting member 371 is formed higher than the second supporting member 373, when the main body 310 is disposed on the ground in a horizontal direction, the first plate 311 may be inclined. Meanwhile, the second plate 313 may be formed in a “⊏” shape, the ends of the “⊏” shape face parts at which the cover unit 350 is connected to the first plate 311, respectively. In other words, the second plate 313 is formed along both lateral edges in a longitudinal direction and one widthwise edge on the top surface of the first plate 311, thereby forming the “⊏” shape.

A mesh-type support film for fixing the gel is inserted into the gel container 330, the cover unit 350 is moved pivotally to be placed at the second position for closing the gel container 330, and then fixed to the second position using the clamping apparatuses 315. From the second position at which the gel container 330 is closed by the cover unit 350 while the supporting member 370 is placed on the ground, a liquid gel is injected into the space between the gel container 330 and the molding plate 353. Since the first supporting member 371 is formed longer than the second supporting member 373, the gel flows on the surface of the first plate 311 to fill the space between the gel container 330 and the molding plate 353. The second supporting member 373 and the third supporting member 375 may be put on the ground, such that the main body 310 of the gel former may be set up on the ground in the vertical direction. After a predetermined time, when the gel in the gel container 330 is hardened, the clamping apparatuses 315 are unfastened to allow the cover unit 350 to be moved back to the first position and to allow the gel container 330 to be open, and then the gel 100 in which the sample injection holes 110 are molded to correspond to the shape of the molding projections 355 is separated from the main body 310, thereby obtaining the gel 100 used in the first analysis method of the present invention as shown in FIG. 2.
(1-2) Sample Injection Step (S2)

Subsequently, a sample to be analyzed is injected into the sample injection holes of the gel prepared as described above (S2). A different sample may be injected into each of the plurality of samples injection holes, and the volume of each sample to be injected may be several microliters, preferably, 0.1 to 5 μl, and more preferably 0.5 to 2 μl. In one exemplary embodiment of the present invention, 1 μl each of different samples was injected into each of 384 grooves among the 404 sample injection holes formed in the gel, and 1 μl of a size marker was injected into each of the remaining 20 sample injection holes.

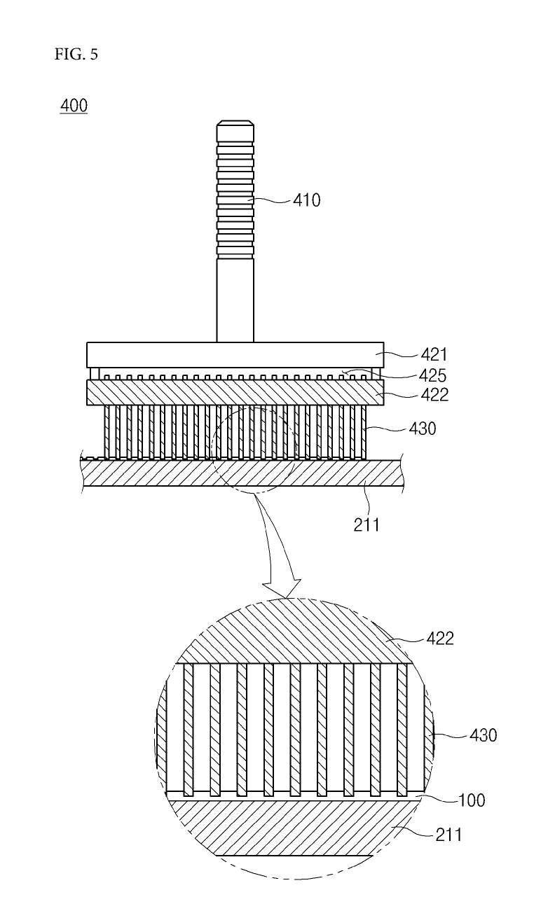

When there are a plurality of sample injection holes formed in the gel as described above, a sample injection unit 400 as shown in FIG. 5 may be used to simultaneously inject multiple samples into the sample injection holes.

Referring to FIG. 5, the sample injection unit 400 includes a rod unit 410, and a second plate 422 connected with a plurality of pins 430 for transferring a sample, and a first plate 421 spaced a predetermined distance from the second plate 422 and guiding the pins 430 so as to be slidable, the set of the plates being connected to the rod unit 410.

The rod unit 410 is formed to facilitate grasping of the sample injection unit 400. The pins 430 are inserted into the sample injection holes of a gel to inject a sample into each of the sample injection holes. Here, since the gel prepared by the first gel former is very vulnerable, when the sample is injected into the sample injection holes formed in the gel, in some cases, the pins 430 may break the gel G. Therefore, as the plurality of pins 430 are formed to slide through the second plate 422 even when applying a predetermined force when the sample is injected into the sample injection holes formed in the gel the gel G is not damaged. In other words, since the first plate 421 is spaced apart from the second plate 422, the plurality of pins 430 may slide in a gap 425 between the first and second plates, and thus the gel G may not be destroyed.
(1-3) Electrophoresis Step (S3)

After the sample is injected into the sample injection holes of the gel as described above, the sample-injected gel is electrophoresed (S3). The electrophoresis is a process of separating proteins of the samples injected into the sample injection holes, and is performed using a horizontal electrophoresis device (not shown). The electrophoresis is performed in a direction perpendicular to a row of the sample injection holes for several minutes, preferably, 10 to 60 minutes, and more preferably 20 to 40 minutes.
(1-4) Transfer Step (S4)

After the electrophoresis is completed as described above, the sample separated in the gel is transferred onto a membrane (S4). The transfer process of the sample may be performed according to a method conventionally used in the art.
(1-5) Antibody Treatment Step (S5)

After the sample is transferred onto the membrane as described above, the membrane is treated with an antibody (S5).

In the first analysis method, the sample-transferred membrane may be treated with one type of antibody. That is, the same antibody may be treated against a plurality of different samples, and therefore, it is possible to simultaneously confirm the absence or presence of a specific protein in the plurality of different samples.

In one embodiment of the present invention, after a total of 384 samples are treated with the same antibody, the absence or presence of specific proteins in the 384 samples was confirmed.

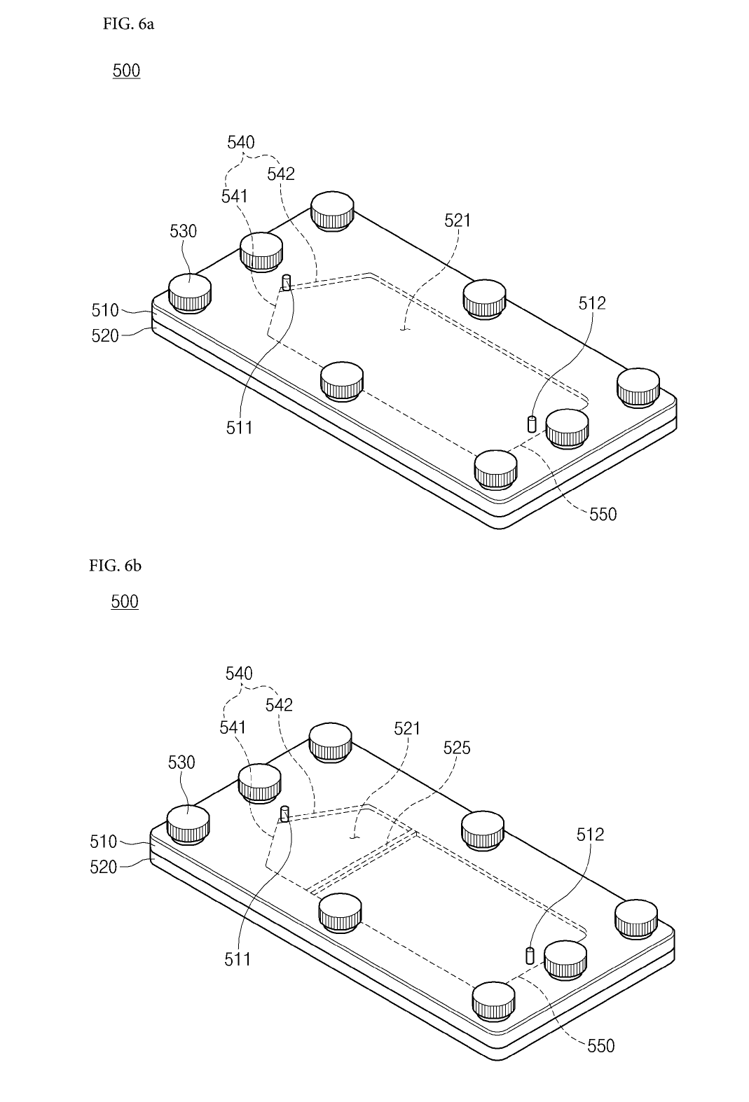

In the first analysis method of the present invention, as shown in FIGS. 6A and 6B, the antibody may be treated using a first antibody incubation chamber 500.

Referring to FIGS. 6A and 6B, the first antibody incubation chamber 500 includes an upper frame 510 in which through holes 512 and 511 are formed and a lower frame 520 having a space 521 where a membrane is placed.

In the upper frame 510, the first through hole 512 through which an antibody is provided and the second through hole 511 through which a waste solution remaining after the reaction is extracted are formed, and when the upper frame 510 is coupled to the lower frame 520, the first and second through holes 512 and 511 in the upper frame 510 communicate with the space 521 in the lower frame 520.

When the sample-transferred membrane is placed in the space 521 communicating with the first and second through holes as described above, the sample transferred onto the membrane reacts with an antibody by injecting a reaction material including the antibody into the first through hole 512, and after the above-mentioned reaction is completed, the waste solution is extracted through the second through hole 511. Afterward, the first and second through holes 512 and 511 may also be used in blocking or washing of the membrane through the injection and discharging of a blocking buffer or washing buffer.

The space 521 has a flat part 550 adjacent to the first through hole 512 at one side and an angled part 540 adjacent to the second through hole 511 at the other side. The angled part 540 is formed by meeting a first side 541 with a second side 542 at a predetermined angle, such that a waste solution or washing buffer solution is gathered at the angled part 540 to be entirely extracted to the outside.

Meanwhile, in the space 521, partitions 525 may be further included as shown in FIG. 6B. The partitions 525 are provided to adjust the volume of the space 521 according to the size of the membrane placed in the space 521, and to reduce the consumption of a sample when antibody treatment has to be performed with a small size of a membrane.
(1-6) Analysis Step (S6)

After the antibody treatment for the membrane is completed, the membrane is analyzed (S6). The analysis process of the membrane may go through exposure and development, or may be performed according to a method conventionally used in the art such as reading data using a fluorescence scanner.

### Second Example

Another aspect of the present invention provides a high-density western blot array analysis method (second analysis method) for simultaneously checking the absence or presence of proteins binding to a plurality of antibodies in a single sample. The second analysis method may be a multi-antibody screening system, and may be actively applied to protein analysis using an antibody library, for example, protein PTM analysis, and therefore several tens to several hundreds of types of antibodies may be simultaneously analyzed as markers.

Referring to FIG. 1, the second analysis method of the present invention also includes preparing a gel (S1), injecting a sample into the gel (S2), electrophoresing the sample in the gel (S3), transferring the sample in the electrophoresed gel onto a membrane (S4), treating the membrane with an antibody (S5), and analyzing the membrane (S6).
(2-1) Gel Preparation Step (S1)

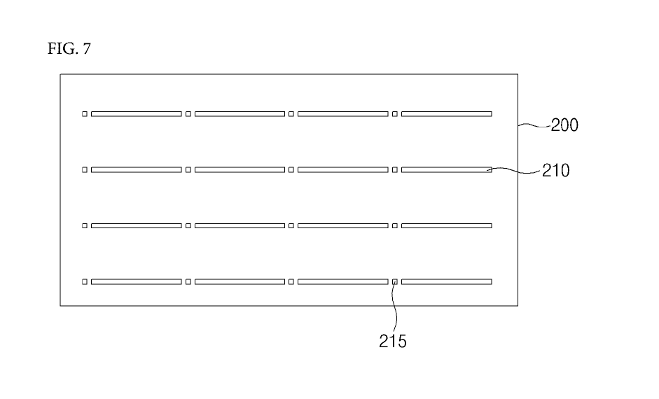

Referring to FIG. 7, in the second analysis method, first, a gel 200 in which one or more sample injection slots 210 each having a predetermined width are arranged in a longitudinal direction of the width so as to be formed in one or more rows is prepared (S1).

Each of the one or more sample injection slots 210 may have a size such that several to several tens of microliters, preferably 0.1 to 50 and more preferably 1 to 20 μl of a sample can be injected.

The sample injection slots 210 have a predetermined width, for example, several tens of mm, preferably 10 to 80 mm, and more preferably 30 to 60 mm.

There may be one sample injection slot 210 formed in a row, or two or more sample injection slots 210 formed in one row. As described above, when a plurality of sample injection slots 210 are formed in one row, the sample injection slots 210 may be arranged at an interval of several mm, preferably 0.5 to 5 mm, and more preferably 1 to 3 mm, and a marker injection hole 215 which can inject a marker may be additionally formed between the sample injection slots 210.

In addition, there may be a plurality of rows consisting of the one or more sample injection slots 210. When there are a plurality of rows, the plurality of rows may be formed to have a wider gap than the distance of electrophoresing the sample injected into each sample injection slot 210, and for example, may be arranged at an interval of several cm, preferably 0.5 to 5 cm, and more preferably 1 to 3 cm.

In one exemplary embodiment of the present invention, four sample injection slots having a width of 45 mm were formed in one row, and a gel having a size of 12 cm×6 cm in which such four rows are arranged at an interval of 1.2 cm was prepared.

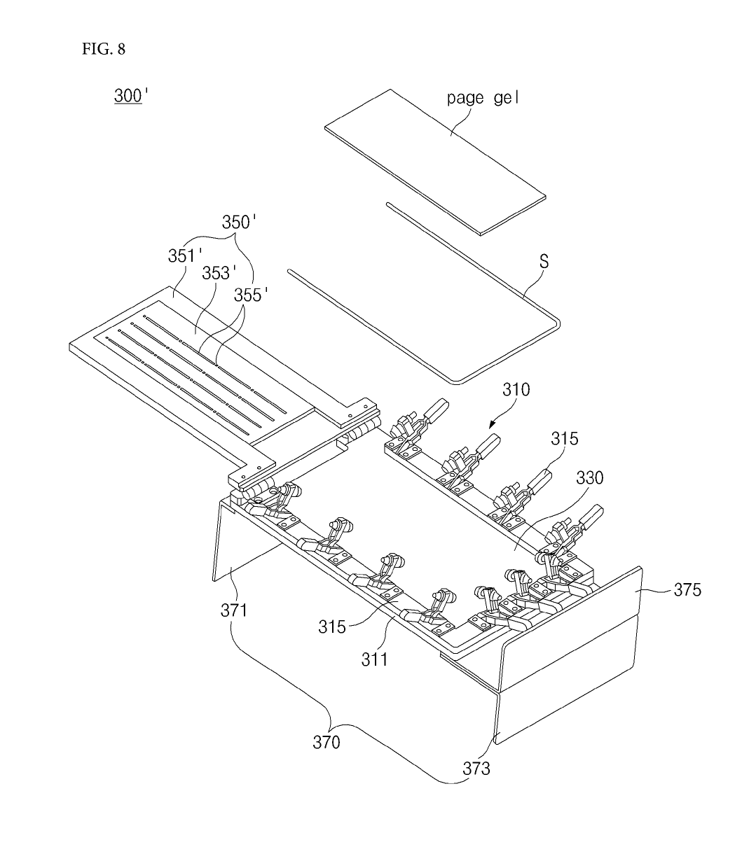

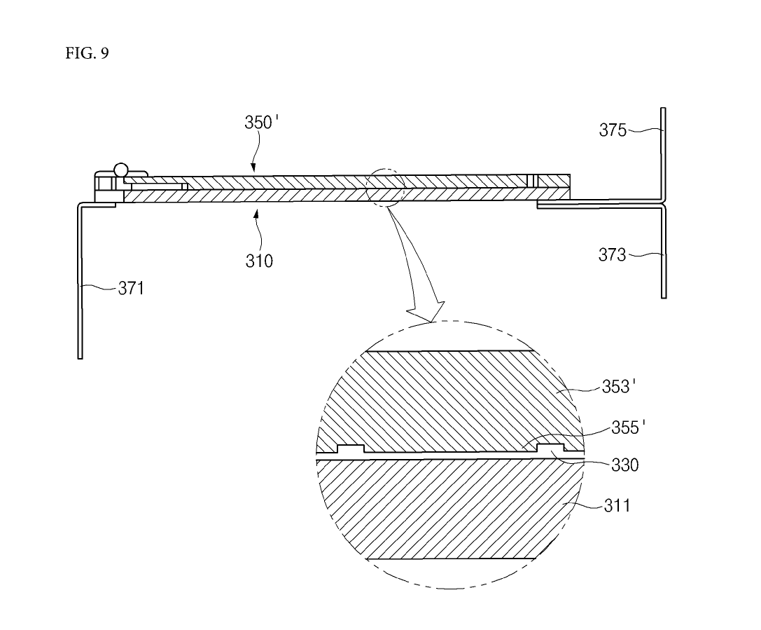

Such a gel 200 used in the second analysis method of the present invention may be prepared using a second gel former 350 as shown in FIGS. 8 and 9.

Referring to FIGS. 8 and 9, the second gel former 350 have the same configuration as the first gel former 300 described in the first example, except the pattern and shape of molding projections 355′ of a molding plate 353′ included in a cover unit 350′. For this reason, the pattern and shape of the molding projections 355′ specific for the second gel former 350 will be explained, and the descriptions of the other components described in the previous description of the first gel former 300 will be omitted.

The molding projections 355′ are formed in a straight line with a width of several tens of millimeters, preferably 10 to 80 mm, and more preferably 30 to 60 mm, such that one or more straight lines are formed in one or more rows at an interval of several mm. When there are a plurality of rows of the molding projections 355′, the rows may be arranged at an interval of several cm. The molding plate 355′ may be integrated with a frame 351′ or is preferably detachable from the frame 351′ so as to be replaced depending on the pattern, gap, size or number of the plurality of molding projections 355′.

In the case of the second gel former 350, a mesh-type support film for fixing the gel is inserted into the gel container 330, the cover unit 350′ is moved pivotally to be placed at the second position for closing the gel container 330, and then fixed to the second position using the clamping apparatuses 315. From the second position at which the gel container 330 is closed by the cover unit 350′ while the supporting member 370 is placed on the floor, a liquid gel is injected into the space between the container 330 and the molding plate 353′. Since the first supporting member 371 is formed longer than the second supporting member 373, the gel flows on the surface of the first plate 311 to fill the space between the gel container 330 and the molding plate 353′. The second supporting member 373 and the third supporting member 375 may be put on the floor, such that the main body 310 of the gel former may be set up on the floor in the vertical direction. After a predetermined time, when the gel in the gel container 330 is hardened, the clamping apparatuses 315 are unfastened to allow the cover unit 350′ to be moved back to the first position and to allow the gel container 330 to be open, and then the gel 200 in which the sample injection slots 210 and marker injection holes 215 are molded to correspond to the shape of the molding projections 355′ is separated from the main body 310, thereby obtaining the gel 200 used in the second analysis method of the present invention.
(2-2) Sample Injection Step (S2)

Subsequently, a sample to be analyzed is injected into the sample injection slots of the gel prepared as described above (S2). A different sample may be injected into each of the plurality of samples injection slots, and the volume of each sample to be injected may be several to several tens of microliters, preferably, 0.1 to 50 μl, and more preferably 1 to 20 μl, and when there are a plurality of sample injection slots, different samples may be injected into the sample injection slots, respectively. In one exemplary embodiment of the present invention, 20 μl of a different sample was injected into each of 16 sample injection slots formed in the gel.

In this second analysis method, since the number of the sample injection slots is not high, unlike the first example, the sample injection unit may not be used, and a common micropipette may be used.
(2-3) Electrophoresis Step (S3)

After the sample is injected into the sample injection slots of the gel as described above, the sample-injected gel is electrophoresed (S3). The electrophoresis is a process of separating proteins of a sample injected into each sample injection slot, and is performed using a horizontal electrophoresis device (not shown). The electrophoresis is performed in a direction perpendicular to a row of the sample injection slots for several minutes, preferably, 10 to 60 minutes, and more preferably 20 to 40 minutes.
(2-4) Transfer Step (S4)

After the electrophoresis is completed as described above, the sample separated in the gel is transferred onto a membrane (S4). The transfer process of the sample may be performed according to a method conventionally used in the art.
(2-5) Antibody Treatment Step (S5)

After the sample is transferred onto the membrane as described above, the membrane is treated with an antibody (S5).

In the second analysis method, the sample isolated from one sample injection slot may be treated with a plurality of different antibodies. That is, as one sample is treated with a plurality of antibodies, the absence or presence of various proteins in one sample may be simultaneously checked.

However, when the sample injection slots are plural, and different samples are injected into the plurality of sample injection slots, respectively, the absence or presence of various proteins in different samples may be simultaneously checked.

Here, the plurality of antibodies may be treated on one sample at a volume of several microliters, preferably, 0.1 to 5 μl, and more preferably, 0.5 to 2 μl.

In one embodiment of the present invention, after one sample is treated with 20 different antibodies, the absence or presence of 20 specific proteins in each of the total of 16 samples was simultaneously checked.

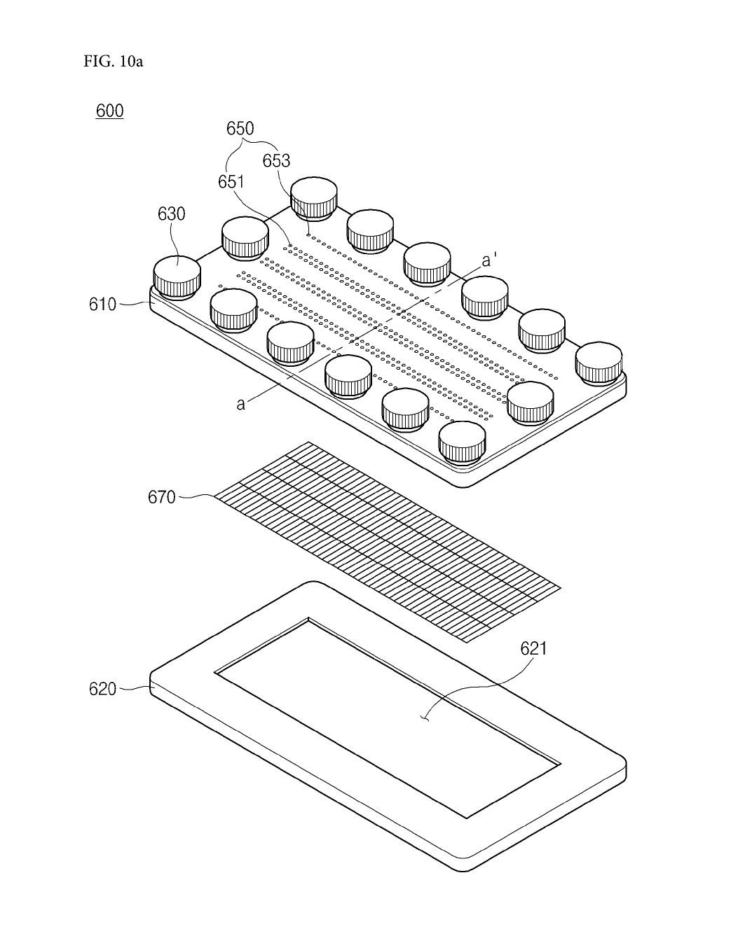

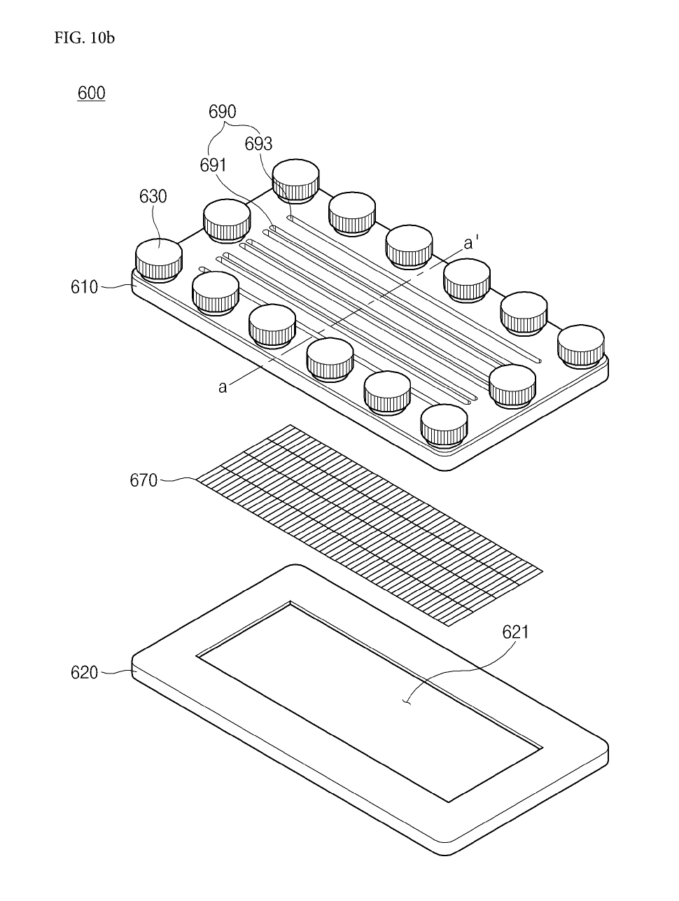

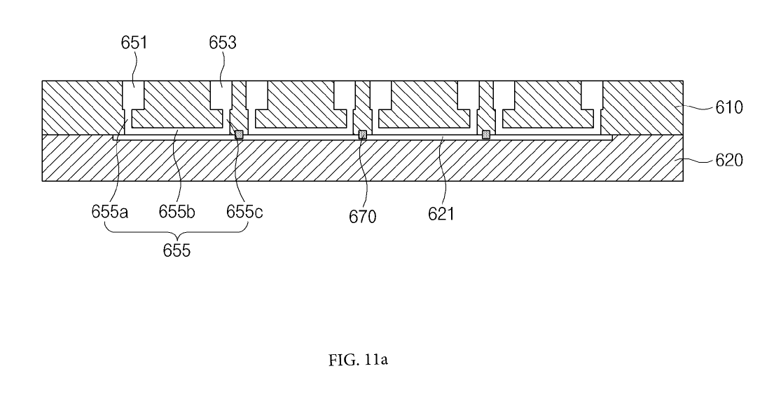

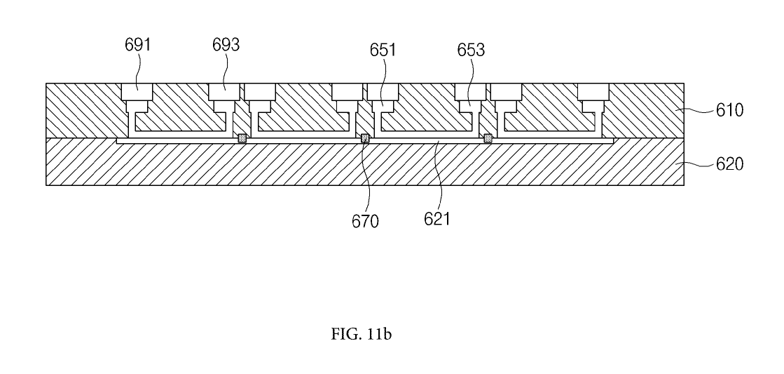

In the second analysis method of the present invention, as shown in FIGS. 10A, 10B and 11, the antibodies may be treated using a second antibody incubation chamber 600.

Referring to FIGS. 10A, 10B and 11, the second antibody incubation chamber 600 includes an upper frame 610 having a plurality of antibody injection holes 651, a plurality of waste solution discharge holes 653 and a plurality of microchannels 655, and a lower frame 620 having a mounting part 621 on which a membrane is placed.

Referring to FIGS. 10A, 10B and 11, the plurality of antibody injection holes 651 and the plurality of waste solution discharge holes 653 are arranged in pairs on the top surface of the upper frame 610 at an interval of several millimeters, preferably 0.5 to 5 mm, and more preferably 1 to 3 mm. 20 or more, preferably 50 or more, and more preferably 100 or more antibody injection holes 651 are arranged in a row, and the antibody injection holes 651 and the waste solution discharge holes 653 may be arranged in pairs in a direction of electrophoresing the sample at an interval of several centimeters, preferably 0.5 to 5 cm, and more preferably 1 to 3 cm. In addition, a plurality of microchannels 655 are included at the lower surface of the upper frame 610. The microchannels 655 include a first microchannel 655a connected with the antibody injection holes 651 in a direction in which the antibody injection holes 651 extend (in a vertical direction), a third microchannel 655c connected with the waste solution discharge holes 653 in a direction in which the waste solution discharge holes 653 extend (in a vertical direction), and a second microchannel 655b formed in a direction perpendicular to the row of the antibody injection holes 651 or waste solution discharge holes 653 to connect the first microchannel 655a with the third microchannel 655c (in a horizontal direction). The first microchannel 655a, the second microchannel 655b and the third microchannel 655 form a “U” shape. Finally, the antibody injection holes 651 and the waste solution discharge holes 653, formed in an upper part of the upper frame 610, are connected with the “U”-shaped microchannels 655 formed in the lower part of the upper frame 610, resulting in the formation of an overall large “U”-shaped channel. To prevent the diffusion of antibodies injected through the antibody injection holes 651 into other microchannels adjacent thereto while passing through the microchannels 655, on the lower surface of the upper frame 610, a sealing member 670 may be additionally formed between the microchannels.

The mounting part 621 on which the membrane is placed is formed in the lower frame 620, and the lower frame 620 is connected with the upper frame 610 such that the lower surface of the upper frame 610 is in close contact with the upper surface of the membrane placed on the mounting part 621. Particularly, if the membrane placed in the mounting part 621 has a predetermined thickness, the mounting part 621 may have a separate space, or if the membrane is very thin and thus substantially needs no separate space, the mounting part 621 may be a certain part present in the lower frame 620, rather than a separately formed space.

In the second antibody incubation chamber 600, fasteners 630 may be additionally formed to firmly fix the coupling between the upper frame 610 and the lower frame 620.

When the sample-transferred membrane is placed in the space of the mounting part 621 of the lower frame 620 and is in close contact with the lower surface of the upper frame 610 (when there is a sealing member 670, being in close contact with the sealing member 670), the sample transferred onto the membrane reacts with an antibody by injecting a reaction material including different types of antibodies into the plurality of antibody injection holes 611, respectively.

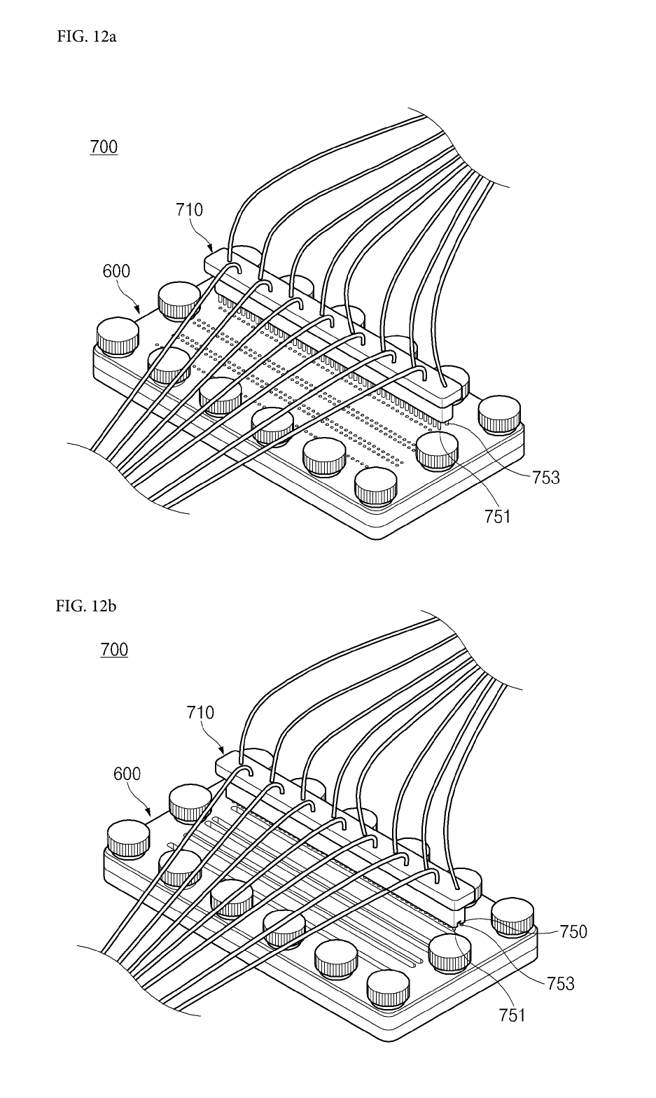

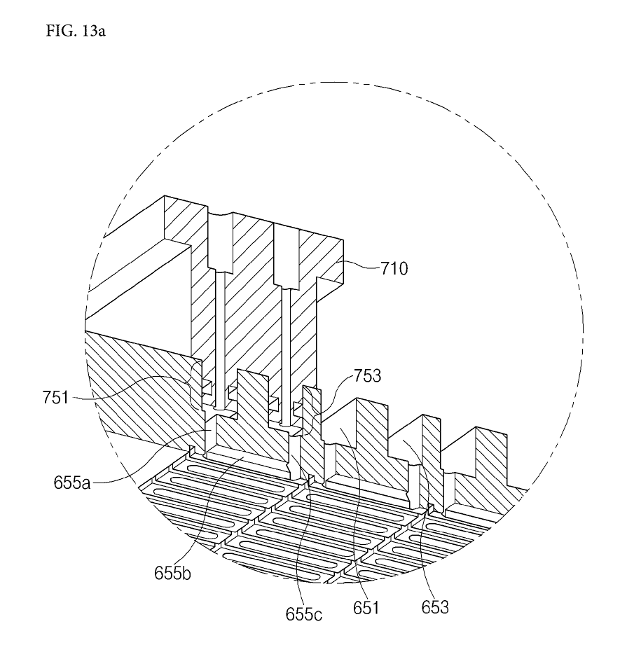

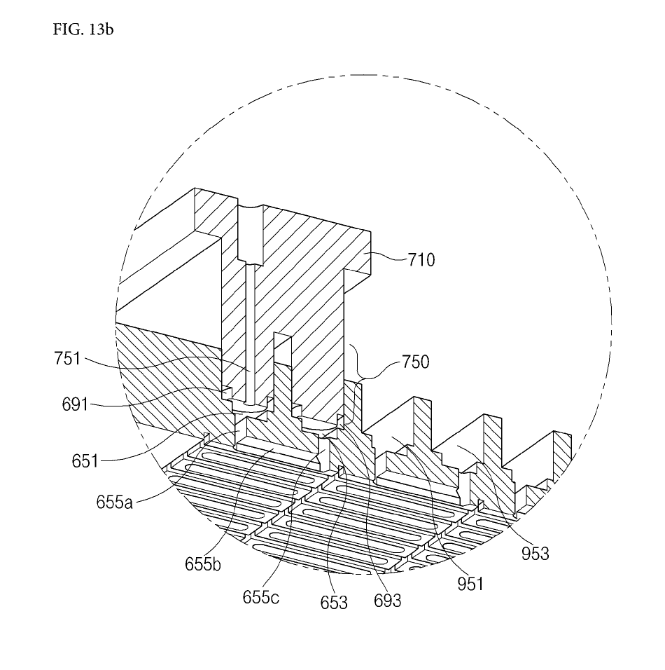

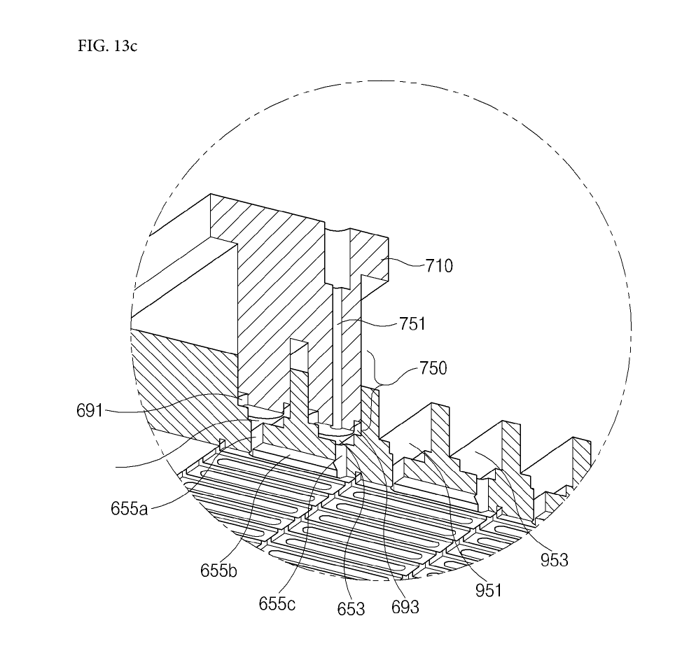

The antibody injection holes 651 and the waste solution discharge holes 653 are used to inject and discharge a washing solution for washing the membrane, as well as the antibodies and a waste solution thereof. The injection of the washing solution into the antibody injection holes 651 and the suction of the waste solution into the waste solution discharge holes 653 are performed by a washing unit 700 as shown in FIG. 12. The washing unit 700 includes injection tubes 751 corresponding to the plurality of antibody injection holes 651 formed in a longitudinal direction of the second incubation chamber 600 and discharge tubes 753 corresponding to the waste solution discharge holes 653. The plurality of injection tubes 751 included in the washing unit 700 are inserted into the antibody injection holes 651 of the second incubation chamber 600, and the plurality of discharge tubes 753 included in the washing unit 700 are inserted into the waste solution discharge holes 653 of the second incubation chamber 600. Each of the plurality of injection tubes 751 is connected with an external washing solution tank (not shown) by a hose, and each of the plurality of discharge tubes 753 is connected with an external vacuum pump (not shown) and a waste solution container (not shown) by a hose.

When an antibody is treated, and a reaction between the treated antibody and the sample in the membrane is completed, the injection tubes 751 and the discharge tubes 753 of the washing unit 700 are inserted into the antibody injection holes 651 and the waste solution discharge holes 653 formed in the upper frame 610, and then, first, a waste solution of the antibody present in the microchannels 655 is sucked through the discharge tubes 753 inserted into the waste solution discharge holes 653 using a vacuum pump (After being sucked as described above, the waste solution is discharged to a waste solution container). Afterward, when the washing solution stored in the washing solution tank is injected into the antibody injection holes 651 through the injection tubes 751, respectively, the washing solution injected as described above flows to the third microchannel 655c through the first microchannel 655a and the second microchannel 655b so as to remove non-binding antibodies present on the membrane. After the washing is completed, a waste solution of the washing solution present in the microchannels 655 is sucked through the discharge tubes 753 inserted into the waste solution discharge holes 653 using a vacuum pump (After being sucked as described above, the waste solution is discharged to a waste solution container).

Meanwhile, in the upper frame 610 of the second antibody incubation chamber 600, as shown in FIG. 10B, fixing grooves 690 to which the washing unit 700 is able to be fixed may be additionally included, and include a first fixing groove 691 formed on the plurality of antibody injection holes 651 and a second fixing grooved 693 formed on the plurality of waste solution discharge holes 653. In this case, the antibody injection holes 651 and the waste solution discharge holes 653 are present in the first and second fixing grooves 691 and 693, respectively.

When the fixing groove 690 are formed in the upper frame 610 of the second antibody incubation chamber 600, the washing unit 700 may include a fixing end 750 which can fix the washing unit 700 by being inserted into the fixing grooves 690, and the injection tubes 751 and the discharge tubes 753 may be included under or may be disposed in the fixing end 750. When the washing unit 700 is inserted into the first and second fixing grooves 691 and 693 and fixed, the numbers of the injection tubes 751 and the discharge tubes 753 of the washing unit 700 may be smaller than those of the antibody injection holes 651 and the waste solution discharge holes 653, and moreover, the injection tubes 751 and the discharge tubes 753 may not be directly inserted into the antibody injection holes 651 and the waste solution discharge holes 653, respectively. In this case, when the washing unit 700 is fixed to the first and second fixing grooves 691 and 693, the injection tubes 751 and the discharge tubes 753 are disposed a predetermined distance apart from the antibody injection holes 651 and the waste solution discharge holes 653 in the first fixing groove 691 and the second fixing groove 693, respectively. The washing solution injected into the injection tubes 751 is injected into the antibody injection holes 651 present in the entire first fixing grooves 691 through the space between the injection tubes 751 and the antibody injection holes 651, and as a sucking power is applied to the entire second fixing groove 693 by a vacuum pump, a waste solution of the washing solution is discharged to the space between the discharge tubes 753 and the waste solution discharge holes 653 present in the entire second fixing groove 693.
(2-6) Analysis Step (S6)

After the antibody treatment for the membrane is completed, the membrane is analyzed (S6). The analysis process of the membrane may go through exposure and development, or may be performed according to a method conventionally used in the art such as reading data using a fluorescence scanner.

The present invention has been explained with reference to exemplary embodiments, but it will be understood by those of ordinary skill in the art that various modifications and variations can be made in the present invention without departing from the spirit and scope of the present invention as described in the accompanying claims.

### EXPLANATION OF REFERENCE NUMERALS

100: gel 110: sample injection hole

200: gel 210: sample injection slot 215: marker injection hole

300: first gel former 300′: second gel former 310: main body

330: gel container 350, 350′: cover unit 370: supporting member

311: first plate 313: second plate 315: clamping apparatus

351: frame 353: molding plate 355: molding projection

351′: frame 353′: molding plate 355′: molding projection

371: first supporting member 373: second supporting member 375: third supporting member

400: injection unit 410: rod unit

421: first plate 422: second plate 430: pin

500: first antibody incubation chamber 525: partition

510: first upper frame 512, 511: first through hole and second through hole

520: first lower frame 521: chamber space

530: first fastener 540: angled part

541: first side 542: second side 550: flat part

600: second antibody incubation chamber

610: upper frame 620: lower frame 621: mounting part

650: hole pair 651: antibody injection hole 653: waste solution discharge hole

655: microchannel 655a, 655b, 655c: first, second and third microchannels

630: second fastener 670: sealing member

690: fixing groove 691: first fixing groove 693: second fixing groove

700: washing unit 710: frame 750: fixing end

751: injection tube 753: discharge tube

S: sealing member

1. A high-density western blot array analysis method for simultaneously checking whether a protein binding to a single antibody is present in a plurality of samples, the method comprising:
preparing a gel in which a plurality of sample injection holes are arranged in one or more rows using a gel former;
injecting a sample into each sample injection hole in the gel;
electrophoresing the sample-injected gel in a direction perpendicular to the row of the sample injection holes;
transferring the sample in the electrophoresed gel to a membrane;
putting the entire sample-transferred membrane into one area formed in an antibody incubation chamber and treating the membrane with an antibody; and
analyzing the antibody-treated membrane.

2. The method according to claim 1, wherein the gel has 50 or more sample injection holes, arranged in one row.

3. The method according to claim 1, wherein the gel has 100 or more sample injection holes, arranged in one row.

4. The method according to claim 2 or 3, wherein the gel has a plurality of rows.

5. The method according to claim 1, wherein the antibody incubation chamber comprises an upper frame in which a first through hole configured to provide an antibody and a second through hole configured to discharge a waste solution are formed;
and a lower frame coupled with the upper frame, in which a sample-transferred space where a membrane is placed is formed and the space communicates with the first through hole and the second through hole of the upper frame.

6. The method according to claim 5, wherein the antibody incubation chamber further includes a partition which adjusts a volume of the space.

7. A high-density western blot array analysis method for simultaneously checking whether proteins binding to a plurality of antibodies are present in a single sample, the method comprising:
preparing a gel in which one or more sample injection slots each having a predetermined width are arranged in a longitudinal direction of the width using a gel former, thereby being formed in one or more rows;
injecting a sample into the sample injection slots in the gel;
electrophoresing the sample-injected gel in a direction perpendicular to the row of the sample injection slots;
transferring the sample in the electrophoresed gel to a membrane;
placing the sample-transferred membrane, to fit the width of the sample injection slots, in an antibody incubation chamber in which a plurality of antibody injection holes and a plurality of microchannels are formed, and then treating the sample transferred onto the membrane along the microchannels with antibodies, respectively, by injecting different types of antibodies into the respective antibody injection holes; and
analyzing the antibodies-treated membrane.

8. The method according to claim 7, wherein the gel has 2 or more sample injection slots, arranged in one row.

9. The method according to claim 8, wherein the gel has a plurality of rows.

10. The method according to claim 7, wherein the antibody incubation chamber includes an upper frame in which a plurality of antibody injection holes and a plurality of waste solution discharge holes are paired and formed in one or more rows on the upper surface thereof, and one end and the other end of each of the plurality of microchannels formed in a direction perpendicular to the row of the antibody injection holes are connected with the antibody injection hole and the waste solution discharge hole, respectively, at the lower surface thereof; and a lower frame in which a mounting part where a sample-transferred membrane is placed is formed, and which is coupled to the upper frame such that the lower surface of the upper frame is in close contact with the upper surface of the membrane mounted in the mounting part.

11. The method according to claim 10, wherein the microchannels formed at the lower surface of the upper frame are formed in a “U” shape.

12. The method according to claim 1, wherein the gel former comprises a main body having a concave gel container for gel injection; and a cover unit which includes a plurality of molding projections which form a plurality of samples injection holes or one or more sample injection slots in a gel injected into the gel container, and is installed to the main body so as to be movable between a first position for opening the gel container and a second position for closing the gel container.

13. The method according to claim 12, wherein, when the cover unit is disposed at the second position, at least some of the molding projections are inserted into the gel injected into the gel container.

14. A gel former for preparing a gel in a western blotting system, comprising:
a main body having a concave gel container for gel injection; and a cover unit which includes a plurality of molding projections which form a plurality of samples injection holes or one or more sample injection slots in a gel injected into the gel container, and is installed to the main body so as to be movable between a first position for opening the gel container and a second position for closing the gel container.

15. The gel former according to claim 14, wherein the cover unit comprises a main body of the cover unit; and a molding plate detachably installed on the main body of the cover unit and including the plurality of molding projections.

16. The gel former according to claim 14, wherein, when the cover unit is disposed at the second position, at least some of the molding projections are inserted into the gel injected into the gel container.

17. An antibody incubation chamber which allows a membrane onto which a protein of an electrophoresed gel is transferred to react with an antibody in a western blotting system, the chamber comprising:
an upper frame in which a first through hole configured to provide an antibody and a second through hole configured to discharge a waste solution are formed; and
a lower frame in which a space where a sample-transferred membrane is placed is formed, and which is coupled to the upper frame while the space communicates with the first through hole and the second through hole.

18. The antibody incubation chamber according to claim 17, further comprising:
a partition for adjusting a volume of the space.

19. An antibody incubation chamber which allows a membrane onto which a protein from an electrophoresed gel is transferred to react with an antibody in a western blotting system, the chamber comprising:
an upper frame in which a plurality of antibody injection holes and a plurality of waste solution discharge holes are paired and formed in one or more rows on the upper surface, and one end and the other end of a plurality of microchannels formed in a direction perpendicular to the row of the antibody injection holes are connected to the antibody injection holes and the waste solution discharge holes at the lower surface; and
a lower frame which has a mounting part where a sample-transferred membrane is placed and which is coupled to the upper frame such that the lower surface of the upper frame is in close contact with the upper surface of the membrane placed in the mounting part.

20. The antibody incubation chamber according to claim 19, wherein the microchannels formed at the lower surface of the upper frame are formed in a “U” shape.

21. The antibody incubation chamber according to claim 19, further comprising:
a first fixing groove formed on the plurality of antibody injection holes and a second fixing groove formed on the plurality of waste solution discharge holes in the upper surface of the upper frame.

22. A sample injection unit for injecting various samples into a plurality of holes, the unit comprising:
a rod unit;
a second plate connected with a plurality of pins configured to transfer the sample, and connected to the rod unit; and
a first plate spaced a predetermined distance apart from the second plate to guide the pins so as to be slidable.

23. A washing unit for washing a plurality of holes and microchannels, comprising:
a plurality of injection tubes corresponding to a plurality of antibody injection holes;
a plurality of discharge tubes corresponding to a plurality of waste solution discharge holes; and
a plurality of hoses which are connected to the injection tubes and the discharge tubes at one end.

24. The washing unit according to claim 23, further comprising a fixing end, and wherein the injection tubes and the discharge tubes are formed under or in the fixing end.
